(19) United States
(12) Patent Application Publication
(10) Pub. No.: US 2007/0288074 A1
(43) Pub. Date: Dec. 13, 2007

(54) Emergency brain cooling device and method of use

(75) Inventor: Karl Cazzini, Orchard Park, NY (US)

Correspondence Address: Kevin D. McCarthy;Roach Brown McCarthy & Gruber, P.C., 1620 Liberty Building, 420 Main Street, Buffalo, NY, 14202, US

(73) Assignee: Gaymar Industries, Inc.

(21) Appl. No.: 11/451,782

(22) Filed: Jun. 13, 2006

(51) Int. Cl. A61F 7/00

(52) U.S. Cl. 607/104

(57) ABSTRACT

The present invention is directed to a scarf having a sealable pocket. Within the pocket is a Joule-Thompson gas chamber having an activation device. When the activation device is triggered, the compressed gas is released from the Joule-Thompson gas chamber through at least one restriction aperture into the sealable pocket. That release results in at least the walls of the sealed pocket being cooled. Prior to being cooled or after being cooled, the scarf is placed over a portion of the patient's body. Preferably, the scarf is positioned over at least one of the patient's carotid arteries, preferably both, to effectively cool the patient's brain and/or body core temperature.

### FIELD OF THE INVENTION

The present invention is directed to a device designed to decrease the temperature of a brain.

### BACKGROUND OF THE INVENTION

It is well known in the medical art that depriving the brain of oxygen for even a short period of time results in irreversible damage to the brain tissue. Such deprivation occurs during stroke, respiratory arrest, cardiac arrest, trauma and other severe bodily disturbances that slow or otherwise hinder the flow of oxygenated blood to the brain. However, it is also known that lowering the temperature of the brain (hypothermia) slows its metabolic activity, and reduces the chance of tissue damage when the oxygen supply is diminished.

At present, operative neurosurgery and cardiac surgery is done in many cases using hypothermia for the specific purposes of maintaining cerebral and cardiac function. In an operating room, this requires use of a cooling module in conjunction with heart/lung bypass techniques by which the patient's blood, and resultantly the patient's brain tissue, is cooled. This widespread ability to rapidly lower brain temperature by as little as four or five degrees can make an enormous difference in preservation of function. However, out in the field, when medical emergencies occur, brain cooling must quickly and expeditiously take place without access to the sophisticated equipment available in the hospital operating room. Various portable brain cooling apparatus are disclosed.

The simplest device to cool the brain is a bag or a scarf-like bag that contains ice cubes, crushed ice or a plastic encapsulated frozen gel. The scarf-like bag apparatus was disclosed in U.S. Pat. No. 5,086,629 to Dibrell. In the past, it was common to place the bag of ice, not the scarf-like bag, on the forehead.

Rather than cooling the brain by the relatively slow heat conduction through the low heat conductivity of the bony skull and hair covering the head, others have promoted alternative methods of cooling the brain. In U.S. Pat. No. 6,405,080, Lasersohn et al. disclosed “It has been discovered that the medical outcome for a patient suffering from severe brain trauma or from ischemia caused by stroke or heart attack is improved if the patient is cooled below normal body temperature (about 37° C.). As understood by the present invention, the medical outcome for many such patients might be significantly improved if the patients were to be mildly or moderately cooled to 32° C.-36° C. relatively quickly for a short period e.g., 1-2 hours, after an ischemic insult. And if desirable, the patient's body temperature can be maintain at about 32° C.-36° C. for approximately 12-72 hours. It is believed that such cooling improves cardiac arrest patient outcomes by . . . improving the mortality rate, in that many organs can benefit from the cooling, and by improving the neurological outcome for those patients that survive. Systems and methods have been disclosed that propose cooling blood flowing to the brain through the carotid artery. An example of such systems and methods is [by Lasersohn et al.]. In particular, Lasersohn et al. taught that various catheters are disclosed which can be advanced into a patient's carotid artery and through which coolant can be pumped in a closed circuit, to remove heat from the blood in the carotid artery and thereby cool the brain. The referenced devices have the advantage over other methods of cooling (e.g., wrapping patients in cold blankets) of being controllable, relatively easy to use, and of being capable of rapidly cooling and maintaining blood temperature at a desired set point.

Lasersohn's device is not practical for emergency situations. It is designed for hospital use in view of the catheter being inserted into the carotid artery. Accordingly, a bag of ice seems to be a practical alternative. Bags of ice, however, are difficult to maintain on a patient. The bags notoriously fall off the patient without adhesives. Using adhesives can be deleterious as well. Some adhesives could cause adverse allergic reactions and/or cause further damage to the patient's skin. Bags of ice have further problems in that ice is a commodity that is difficult to transport and maintain for extensive periods of time in an emergency like setting, like an ambulance.

In response to those problems, Schwartz discloses in U.S. Pat. No. 5,916,242, the use of a light weight, easily applied neck encircling collar in firm contact with the soft tissue of the neck, and particularly in good thermal contact with the carotid arteries traversing the neck. A coolant flowing through channels embedded in the collar rapidly cools the blood flowing through the carotid arteries which branch into blood vessels throughout the brain providing vascular access and attendant rapid internal cooling throughout the brain including its deepest recesses. Placing the collar on the patient's neck is easily and quickly accomplished simultaneously with other emergency medical techniques, such as CPR, which maintain the patient's heart and lung activity. The collar of the invention contains no metallic parts; the collar, including the coolant channel, may be non-metallized fabric or plastic. This allows X-ray, CT scan, or MRI procedures to be used while the collar is in place without impairing the effectiveness of the procedure.

In particular, Schwartz wrote (without reference numerals) “a substantially circular collar containing a gap, has a channel running about the circumference of the collar. The ends of the channel are sealed, leaving the gap in the collar. Inlet tube and outlet tube, located proximate to the ends serve as entrance and exit for a coolant flowing through the collar in channel. The collar's height “X” is sufficient to cover a large portion of the carotid artery in the neck of a patient having the collar in place. A fastener, such as a Velcro strip, is used to firmly secure the collar about the neck of the patient. The collar is fabricated from either a fabric or plastic having a good thermal transfer coefficient, and capable of sustaining coolant fluid flow through the channel without leakage. . . . The collar is in contact with the carotid artery substantially over the full distance where the carotid artery traverses the neck. The collar is firmly secured in position against the skin of the neck, and is in solid contact over the carotid artery. Coolant flows from the refrigerant supply via the inlet tube through the channel of the collar and back down to the refrigerant supply via the outlet tube, cooling the carotid artery as well as other vascular vessels in the neck, and (sic) attendantly the brain.”

Schwartz discloses a collar system that needs to be interconnected to an exterior refrigerant supply. This collar system is not adaptable for immediate emergency responses.

A portable cooling device is disclosed in U.S. Pat. No. 5,261,399, issued in the names of Klatz et al. The device is for use on an injured or disabled patient. Klatz et al. disclose a helmet and back plate containing cavities in which a coolant flows to cool the brain by means of heat conduction through the skull and upper spinal column. This device is bulky is difficult to place over and under a patient.

The present invention solves these problems for immediate and efficient emergency cooling of the brain.

### SUMMARY OF THE INVENTION

The present invention is directed to a scarf having a sealable pocket. Within the pocket is a Joule-Thompson gas chamber having an activation device. When the activation device is triggered, the compressed gas is released from the Joule-Thompson gas chamber through at least one restriction aperture into the sealable pocket. That release results in at least the walls of the sealed pocket being cooled. Prior to being cooled or after being cooled, the scarf is placed over a portion of the patient's body. Preferably, the scarf is positioned over at least one of the patient's carotid arteries, preferably both, to effectively cool the patient's brain and/or body core temperature.

### BRIEF DESCRIPTION OF THE DRAWINGS

FIG. 1 illustrates an embodiment of the present invention.

FIG. 2 illustrates a cross-sectional view of FIG. 1 taken along lines 2-2.

FIG. 3 illustrates an alternative embodiment of the present invention.

### DETAILED DESCRIPTION OF THE INVENTION

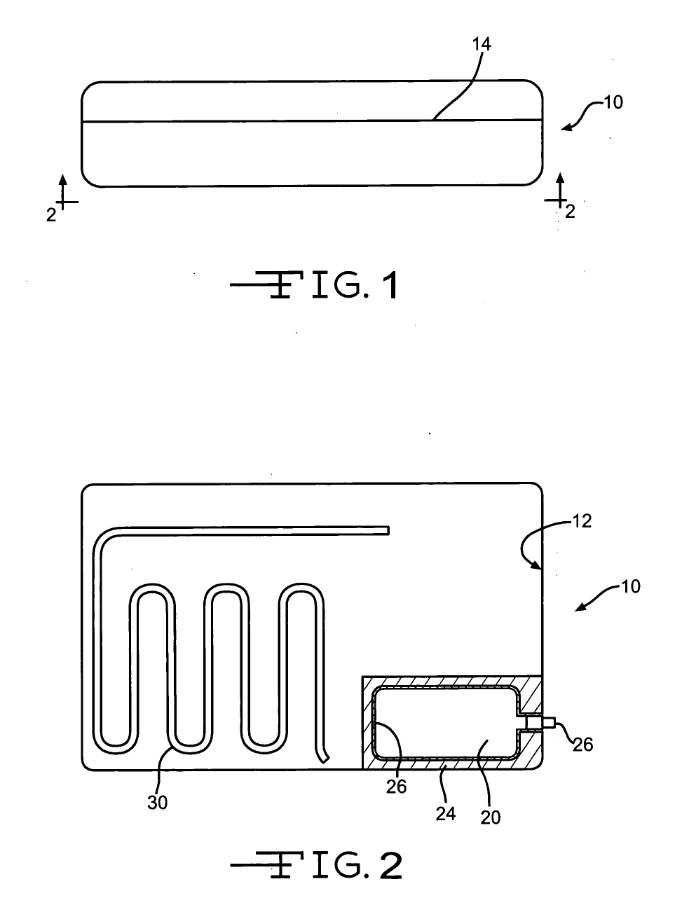

FIG. 1 depicts an embodiment of the cooling mechanism 10. The cooling mechanism looks like a scarf. The scarf 10 can be applied to any part of a patient's body, but is usually applied to the neck of the person desiring either cooling after or during exercise. The scarf 10 is preferably formed from any material that allows the efficient transfer of thermal energy to a patient without losing its attribute of being a cooling mechanism that can easily wrap around a patient's body part, preferably the neck. Examples of the material for the cooling mechanism include and are not limited to cotton toweling material, conductive polymer material, natural fibers, polymeric material, polymeric material with rivet like devices interspaced therein, and flexible metallic material. For some of these materials, there may be an insulation layer (not shown) within the interior chamber 12 to ensure that the desired cold thermal temperature of the cooling mechanism 10 is maintained.

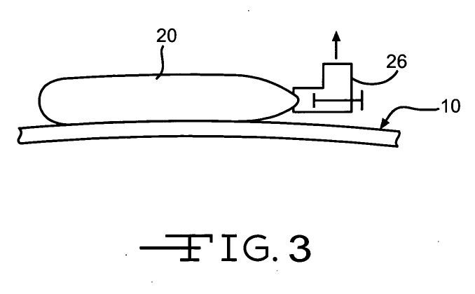

Within the cooling mechanism 10 is an interior chamber 12 as illustrated in FIG. 2. The interior chamber 12 can be sealed by a flap 14, as illustrated in FIG. 1. The flap 14 seal the interior chamber 12 from the exterior environment by numerous methods. Some of the methods include and are not limited to adhesives, hook and loop systems, heat sealing, sonic sealing, buttons, zipper, tongue and groove system, and snap systems. Alternatively, the flap 14 can be (1) permanently sealed to separate the exterior environment from the chamber; or re-sealable to allow a user to open and close, once or numerous times, the flap to remove or insert components into the chamber.

Within the interior chamber 12 is a Joule-Thompson container 20 as illustrated in FIG. 2. The container 20, when not activated or depleted, contains a high pressure gas. The high pressure gas can be any gas that abides to the Joule-Thompson effect of cooling surrounding devices in the interior chamber 12 upon being properly released from the container 20. Examples of the pressurized gas include and are not limited to CO₂, N₂O, nitrogen, argon, or a mix of argon and nitrogen due to their thermodynamic properties, and their inertness.

The gas that becomes the pressurized gas within the gas container 20 is initially compressed then the temperature of the gas is lowered using a pre-cooler or any conventional heat exchanger to reduce the temperature. The gas is now in a state of high pressure and relatively low temperature. This technique is a conventional method for preparing a gas for a throttle-type Joule-Thompson effect device, and is known to those of ordinary skill in the art.

The pressurized gas is released from the container 20 through at least one restriction aperture 22. The restriction aperture 22 directs the pressurized gas into the remainder of the interior chamber 12. When the pressurized and relatively low temperature gas is passes through the restriction aperture, which can be a conventional throttling valve, the gas expands. The expansion of the gas results in an isenthalpic reduction in pressure that causes a large drop in temperature in the interior chamber 12.

It is desired that the at least one restriction aperture directs a portion of the thermal energy generated by the Joule-Thompson effect of the pressurized gas to a heat sink/exchange system 24. The system 24 radiates the energy generated by the Joule-Thompson effect to the remainder of the interior chamber 20. In one embodiment, the system 24 surrounds the container 20. One reason for the system 24 surrounding the container 20 is to retain the container 20 in place. Another reason for the system surrounding the container 20 is to ensure the thermal energy from the pressurized gas, when released from the container 20, is properly distributed throughout the interior chamber 12.

An activation device 26 is the device used to properly release the pressurized gas from the container 20 to the remainder of the interior chamber 12. The activation device is attached to the container 20. The activation device is a switch, a button, or any other type of conventional device that can be used to trigger the release of the pressurized gas from the container 20 through the throttling valve (restriction aperture) 22 to the remainder of the interior chamber 12. The activation device 26 is positioned near the exterior surface of the interior chamber 20, or protrudes from the interior chamber 20. That way, the activation device 26 can be easily found when needed.

In one embodiment of the present invention, the remainder of the interior chamber 20 is empty. That embodiment, however, does not ensure that the large drop in temperature will remain in the interior chamber. To maintain the large drop in temperature, the interior chamber 12 can be filled with a conventional cooling liquid, a cooling pad 30, or combinations thereof. The cooling pad 30 can be a gel or a super-absorbent polymer.

The present invention is designed for reducing the temperature of a patient in a non-hospital and sometimes, non-ambulance, environment. When the patient is being treated, the patient is not always positioned where ice can be easily obtained, or positioned to obtain the desired pressurized gas. The present invention is directed to a cooling mechanism that is all inclusive. That means the user of the cooling mechanism need not interconnect the cooling mechanism to another device to have it properly operate on the patient.

To operate the cooling mechanism, the user activates the activation device which initiates the Joule-Thompson effect of cooling the cooling mechanism. The cooling mechanism can be positioned on the patient either before or after the activation device is activated.

If the flap is re-sealable, the cooling mechanism can be reused. The container 20 can be refilled with a pressure gas and the cooling mechanism can be reused. Alternatively, the container 20 can be disposed, and replaced with a new container 20.

The cooling mechanism is not designed to be used in a hospital or a critical care unit because hospitals and the like normally have advanced cooling systems wherein the temperature applied to the patient can be maintained with greater control.

In another embodiment, the gas container 20 can be positioned directly upon the patient's skin, or positioned over the patient's skin with a substrate, like the scarf material 10, positioned between the patient's skin and the gas container 20 as illustrated in FIG. 3. The latter alternative embodiment is preferred over the former alternative embodiment to decrease the chance of damaging the patient's skin. The gas container 20 can have the gas released through the activation device 26 to the environment, as illustrated in FIG. 3 with the arrow, in a predetermined rate to provide the desired temperature.

Although the invention has been described in terms of specific embodiments, it should be understood that this is by way of illustration only and that the invention is not necessarily limited thereto, since alternative embodiments will become apparent to those skilled in the art as a result of this disclosure.

We claim:

1. A flexible cooling apparatus comprising
a flexible material having sufficient length to at least contact a desired portion of a patient's body;
a chamber within the flexible material;
a Joule-Thompson container having a pressurized gas and being attached to the flexible material;
a throttling valve that directs the pressurized gas from the Joule-Thompson container into the chamber;
an activating device positioned over the flexible material and controls the operation of the throttling valve;
wherein when the activating device is triggered to operate the flexible cooling apparatus, the pressurized gas is released from the Joule-Thompson container into the chamber where the gas reduces the temperature in the chamber.

2. The apparatus of claim 1 wherein the Joule-Thompson container is within the chamber.

3. The apparatus of claim 1 wherein the flexible cooling apparatus is positioned over at least one carotid artery of a patient.

4. The apparatus of claim 1 wherein the flexible cooling apparatus is positioned over both carotid arteries of a patient.

5. The apparatus of claim 1 further comprising a flap that separates the chamber from the exterior environment.

6. The apparatus of claim 5 wherein the flap is resealable and allows a user to insert or remove items from the chamber.

7. The apparatus of claim 5 wherein the flap permanently seals the chamber from the exterior environment.

8. The apparatus of claim 2 wherein a heat sink surrounds the Joule-Thompson.

9. The apparatus of claim 1 wherein the Joule-Thompson container is re-fillable with gas.

10. The apparatus of claim 1 wherein the Joule-Thompson container is disposable.

11. The apparatus of claim 1 wherein the flexible cooling apparatus is disposable.

12. The apparatus of claim 1 further comprising a cooling pad in the chamber.

13. The apparatus of claim 1 further comprising a cooling fluid in the chamber.

14. The apparatus of claim 1 further comprising a cooling fluid and a cooling pad in the chamber.

15. The apparatus of claim 1 wherein the wherein the flexible cooling apparatus is a self-contained device.

16. The apparatus of claim 1 wherein the gas is selected from the group consisting of CO₂, N₂O, nitrogen, argon, or a mix of argon and nitrogen.

17. A method of using a flexible cooling apparatus comprising
triggering an activating device interconnected to a throttling valve that directs a pressurized gas from a Joule-Thompson container, that is attached to a flexible material having sufficient length to at least contact a desired portion of a patient's body, to a chamber within the flexible material where the gas reduces the temperature in the chamber;
applying the flexible cooling apparatus to a patient's body to cool the patient.

18. The method of claim 17 wherein the Joule-Thompson container is within the chamber.

19. The method of claim 17 wherein the flexible cooling apparatus is positioned over at least one carotid artery of a patient.

20. The method of claim 17 wherein the flexible cooling apparatus is positioned over both carotid arteries of a patient.

21. The method of claim 17 further comprising a flap that separates the chamber from the exterior environment.

22. The method of claim 21 wherein the flap is resealable and allows a user to insert or remove items from the chamber.

23. The method of claim 21 wherein the flap permanently seals the chamber from the exterior environment.

24. The method of claim 18 wherein a heat sink surrounds the Joule-Thompson.

25. The method of claim 17 wherein the Joule-Thompson container is re-fillable with gas.

26. The method of claim 17 wherein the Joule-Thompson container is disposable.

27. The method of claim 17 wherein the flexible cooling apparatus is disposable.

28. The method of claim 17 further comprising a cooling pad in the chamber.

29. The method of claim 17 further comprising a cooling fluid in the chamber.

30. The method of claim 17 further comprising a cooling fluid and a cooling pad in the chamber.

31. The method of claim 17 wherein the wherein the flexible cooling apparatus is a self-contained device.

32. The method of claim 17 wherein the gas is selected from the group consisting of CO₂, N₂O, nitrogen, argon, or a mix of argon and nitrogen.

33. A flexible cooling apparatus comprising
a flexible material having sufficient length to at least contact a desired portion of a patient's body;
a Joule-Thompson container having a pressurized gas and being positioned over the flexible material;
a throttling valve that directs the pressurized gas from the Joule-Thompson container into the chamber;
an activating device positioned over the flexible material and controls the operation of the throttling valve;
wherein when the activating device is triggered to operate the flexible cooling apparatus, the pressurized gas is released from the Joule-Thompson container into the environment where the gas reduces the temperature in the container.
